(19) United States
(12) Patent Application Publication
(10) Pub. No.: US 2022/0163370 A1
(43) Pub. Date: May 26, 2022

(54) Analytical Toilet with Pressure Sensor

(71) Applicant: Medic, Inc., Provo, UT (US)

(72) Inventors: David R. Hall, Provo, UT (US); Joshua Larsen, Spanish Fork, UT (US); John W. Christiansen, American Fork, UT (US)

(21) Appl. No.: 17/535,461

(22) Filed: Nov. 24, 2021

Related U.S. Application Data
(60) Provisional application No. 63/117,617, filed on Nov. 24, 2020.

(51) Int. Cl. G01G 17/04; G01G 19/52; E03D 9/00

(52) U.S. Cl. CPC G01G 17/04; A61B 5/208; E03D 9/00; G01G 19/52

(57) ABSTRACT

An analytical toilet, comprising a bowl adapted to receive excreta; a source of refill water to the toilet controlled by a refill valve; a P-trap with a height over two inches in a drain from the toilet; a pressure sensor in fluid communication with the bowl; and a controller in communication with the pressure sensor and the refill valve; wherein the controller monitors water level in the toilet following a flush and controls the valve so that the toilet is filled to a water level no more than two inches in the P-trap is disclosed. An analytical toilet comprising a bowl adapted to receive excreta; a pressure sensor in fluid communication with the bowl; a drain valve adapted to allow or disallow flow from the bowl to a drain; and a controller in communication with the pressure sensor and the drain valve is disclosed.

### CROSS-REFERENCE TO RELATED APPLICATIONS

This application claims the benefit under 35 U.S.C. 119(e) of U.S. Provisional Patent Application No. 63/117,617 titled “Sealed Sewer with Pressure Transducer for Uroflowmetry” filed on 24 Nov. 2020, which disclosure is incorporated herein by reference in its entirety.

### TECHNICAL FIELD

The present disclosure relates to analytical toilets. More particularly, it relates to analytical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Uroflow metrics (uroflowmetry) are one type of valuable data that may be collected by an analytical toilet. Uroflowmetric data may reveal several issues related to kidney and prostate health. However, accurate data is difficult to obtain in a toilet environment, particularly when trying to obtain the data in a conventional toilet.

Just a few examples of smart toilets and other bathroom devices can be seen in the following U.S. Patents and Published Applications: U.S. Pat. No. 9,867,513 entitled “Medical Toilet With User Authentication”; U.S. Pat. No. 10,123,784 entitled “In Situ Specimen Collection Receptacle In A Toilet And Being In Communication With A Spectral Analyzer”; U.S. Pat. No. 10,273,674 entitled “Toilet Bowl For Separating Fecal Matter And Urine For Collection And Analysis”; US 2016/0000378 entitled “Human Health Property Monitoring System”; US 2018/0020984 entitled “Method Of Monitoring Health While Using A Toilet”; US 2018/0055488 entitled “Toilet Volatile Organic Compound Analysis System For Urine”; US 2018/0078191 entitled “Medical Toilet For Collecting And Analyzing Multiple Metrics”; US 2018/0140284 entitled “Medical Toilet With User Customized Health Metric Validation System”; and US 2018/0165417 entitled “Bathroom Telemedicine Station.” The disclosures of all these patents and applications are incorporated by reference in their entireties.

### SUMMARY

In a first aspect, the disclosure provides an analytical toilet, comprising a bowl adapted to receive excreta; a source of refill water to the toilet controlled by a refill valve; a P-trap with a height over two inches in a drain from the toilet; a pressure sensor in fluid communication with the bowl; and a controller in communication with the pressure sensor and the refill valve; wherein the controller monitors water level in the toilet following a flush and controls the valve so that the toilet is filled to a water level no more than two inches in the P-trap.

In a second aspect, the disclosure provides an analytical toilet comprising a bowl adapted to receive excreta; a pressure sensor in fluid communication with the bowl; a drain valve adapted to allow or disallow flow from the bowl to a drain; and a controller in communication with the pressure sensor and the drain valve.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a side cross-sectional view of a first exemplary embodiment of a toilet system according to the present disclosure.

FIG. 2 is a side cross-sectional view of a second exemplary embodiment of a toilet system according to the present disclosure.

FIG. 3 is a perspective view of an exemplary embodiment of a gate valve for the embodiment of FIG. 2.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, “uroflowmetry” is meant to refer broadly to the measurement of urine flow over time by any means, directly or indirectly.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, transducers, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “data connection” and similar terms are meant to refer to any wired or wireless means of transmitting analog or digital data and a data connection may refer to a connection within a toilet system or with devices outside the toilet.

### Exemplary Embodiments

The present disclosure relates to systems and mechanisms for measuring uroflowmetry data in a toilet. One method of measuring uroflowmetry is to measure the level of liquid in the toilet bowl (e.g., with a proximity sensor). However, this is difficult to do accurately as a precise knowledge of bowl and drain geometry, which may not be possible. In particular, conventional porcelain toilets of the same manufacture may vary considerably. The present disclosure discusses the use of pressure sensor to measure the pressure in the toilet plumbing which is proportional to volume.

The present disclosure describes a system that preferably interacts with the toilet only at the exit from the toilet into the sewer. This means that a ceramic toilet/bowl can be used with little or no modification since no sensor or valve etc. is required to interact with the bowl or p trap before the exit.

Due to regulatory requirements, the P-traps in conventional toilets are designed with a two-inch water barrier to prevent backflow of sewer gasses through the toilet. This barrier is maintained by overfilling the toilet after flushing so that the water level in the P-trap is at the overflow level.

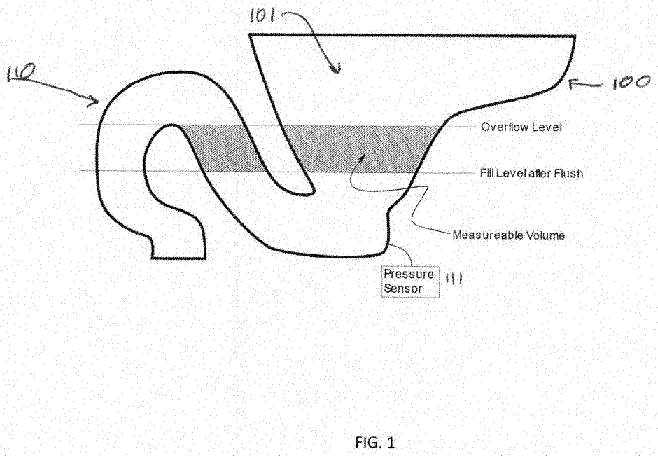

Now referring to FIG. 1, an exemplary embodiment of uroflow measurement system for an analytical toilet 100 is shown. An oversized P-trap 110 allows for the maintenance of the required two-inch water barrier without the bowl 101 being filled to the overflow level. In a preferred embodiment, a pressure sensor or other sensor is used to monitor water level after a flush and shut off flow to the toilet once the desired level is reached. In a preferred embodiment, the P-trap provides four inches of height. The bottom two inches are filled with water forming the required water barrier. The top two inches allow for the level of the toilet to increase during use without reaching the overflow volume.

In various exemplary embodiments, a pressure sensor 111, which may be the same or different from the sensor used to control refill, is provided in the bowl or P-trap. The addition of excreta to the toilet causes the pressure measurement to increase proportionally to the additional mass. Data from the pressure sensor is used to track the weight of the bowl contents over time and identify excreta events, classify excreta events, and calculate mass and volumetric excreta profiles for an event.

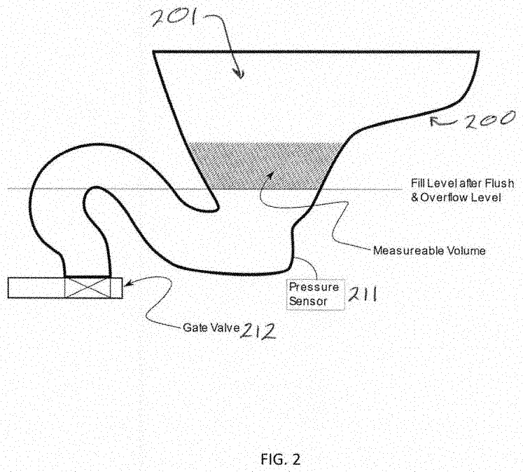

Now referring to FIG. 2, another exemplary embodiment of a toilet 200 and uroflow measurement system is shown. A valve 212 is placed at the outlet from the toilet to the sewer floor flange. A pressure transducer 211 is located upstream from the valve. The pressure valve 211 may be located in the bowl or in the outlet (e.g., a P-trap). The pressure at the transducer is a function of the level of the toilet contents. During a urination event, the volume is increased which increases the pressure on the transducer. This data can be used to track bowl volume over time and to calculate uroflowmetric data and a urination profile. In various exemplary embodiments, the pressure sensor may be, but is not limited to, a potentiometric, inductive, capacitive, piezoelectric, strain gauge, or variable reluctance pressure sensor. In various preferred exemplary embodiments, the pressure sensor is capable of functioning in both a liquid and gaseous environment.

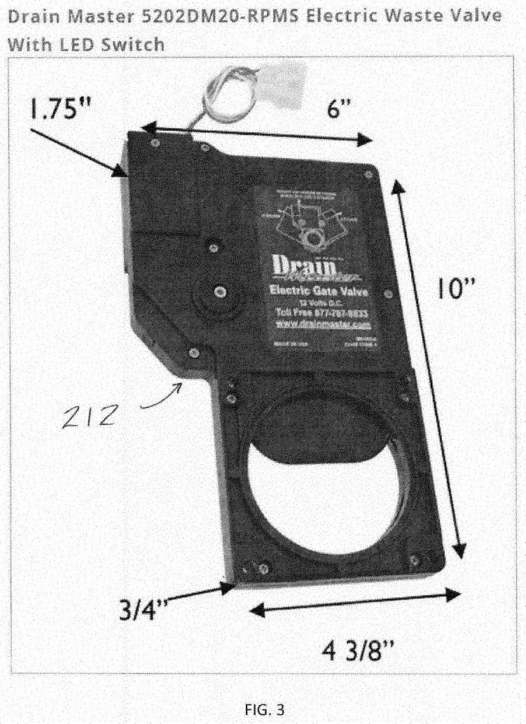

In various exemplary embodiments, any type of valve may be used as the drain valve 212. Referring to FIG. 3, an exemplary embodiment of a gate valve used in a preferred embodiment of the uroflowmetric system is shown. The drain valve is preferably positioned as shown in FIG. 2. Water overflowing from the bowl into the drain is retained in the system by the valve until after the completion of the excretion event. The valve is preferably opened prior flushing the bowl to prevent back of flush liquid in the bowl.

After completing a flush cycle, a new baseline pressure may be established based on the level of liquid remaining in the toilet. Because a new baseline is established prior to each use, it is immaterial whether the amount of post-flush liquid is exactly the same from flush to flush.

Placing the valve and sensor at the exit from the toilet limits interaction with the toilet to a single interface. In a preferred embodiment, the interface may be a flexible rubber joint or seal that may be clamped in place or compliantly stretched to form a seal and hold the components together. The valve/sensor system would also attach to the sewer floor flange in the same way that a conventional toilet would be attached. In an alternative embodiment, the valve/sensor system may be part of a platform installed between the toilet and the floor.

Placing a complete seal between the toilet and the sewer drain eliminates the need to drain the toilet below its overflow point. It also makes it possible to measure uroflow data after the toilet reaches its overflow point. Even if the toilet bowl reaches and exceeds its overflow volume, very little liquid will flow onto the valve and air in the pipe will not escape into the bowl. Thus, accurate pressure readings can still be taken after the bowl reaches overflow volume.

In various exemplary embodiments, before the user deposits any waste into the toilet, the valve would be electrically actuated closed. This would create a trapped air bubble that will increase in pressure as more substance is deposited into the bowl. On a flush trigger, or if the bowl is risking overflow, the valve would open and allow for a normal siphonic toilet flush into the sewer.

In various preferred embodiments, the pressure transducer is placed as close to the electronic valve as possible to avoid needing to interact in an additional point with the ceramic, but it could be placed anywhere in the “air bubble”. After a reading is taken, the path between the pressure transducer and the “air bubble” could be flushed out with water. This would prevent any potential contamination in the toilet from clogging or blocking the pressure path.

In various exemplary embodiments, the valve on the toilet would remain in its open position except for when a user is using the toilet. This would prevent most issues with the valve remaining closed upon power failure. Alternatively, a battery backup powerful enough just to open the valve could be used. Ideally the valve would also be equipped with a manual way to open it. For example, the valve in FIG. 3 has a screw point that could open the valve in case of electric failure.

Although the disclosed systems and methods have been shown in connection with a conventional toilet, the system is not limited to conventional toilets. The system may be adapted for use with wall mount toilets and urinals by including a spacer or platform for those that includes the disclosed valve and sensor system. A valve and sensor could also be incorporated into the designs of other toilet and urinal types.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet, comprising:
a bowl adapted to receive excreta;
a source of refill water to the toilet controlled by a refill valve;
a P-trap with a height over two inches in fluid communication with a drain from the toilet;
a pressure sensor in fluid communication with the bowl; and
a controller in communication with the pressure sensor and the refill valve;
wherein the controller controls the water level in the toilet following a flush by monitoring water level and controlling the valve so that the refill valve is closed at a water level no more than two inches in height in the P-trap; and
wherein data from the pressure sensor is used by the controller to measure changes to the mass of the contents of the bowl.

2. The toilet of claim 1 further comprising a drain valve adapted to allow or disallow flow from the bowl to the drain.

3. The toilet of claim 2 wherein the drain valve is a gate valve.

4. The toilet of claim 2 wherein the drain valve is attached to the sewer floor flange.

5. The toilet of claim 1 wherein the pressure sensor is a pressure transducer.

6. The toilet of claim 1 wherein the drain valve is connected to the toilet outlet.

7. The toilet of claim 1 wherein the pressure sensor is located in a bottom area of the bowl.

8. The toilet of claim 1 wherein the pressure sensor is located in the P-trap.

9. The toilet of claim 1 further comprising a level sensor for detecting the level of water in the P-trap in communication with the controller.

10. The toilet of claim 1 further comprising a data connection between the controller and a data storage device.

11. The toilet of claim 1 wherein the height of the P-trap is about four inches.

12. An analytical toilet comprising:
a bowl adapted to receive excreta;
a pressure sensor in fluid communication with the bowl;
a drain valve controlling flow out of the drain; and
a controller in communication with the pressure sensor and the drain valve.

13. The toilet of claim 12 wherein the conduit comprises a p-trap.

14. The toilet of claim 12 wherein the drain valve is a gate valve.

15. The toilet of claim 12 wherein the drain valve is attached to the sewer floor flange.

16. The toilet of claim 12 wherein the pressure sensor is a pressure transducer.

17. The toilet of claim 12 wherein the drain valve is connected to the toilet outlet.

18. The toilet of claim 12 wherein the pressure sensor is located in a bottom area of the bowl.

19. The toilet of claim 12 wherein the pressure sensor is located in a P-trap.

20. The toilet of claim 12 further comprising a data connection between the controller and a data storage device.
